(19) United States
(12) Patent Application Publication
(10) Pub. No.: US 2016/0045319 A1
(43) Pub. Date: Feb. 18, 2016

(54) ACETABULAR CUP PROSTHESIS

(71) Applicant: Derek James Wallace McMinn, West Midlands (GB)

(72) Inventor: Derek James Wallace McMinn, West Midlands (GB)

(21) Appl. No.: 14/713,040

(22) Filed: May 15, 2015

(30) Foreign Application Priority Data
Aug. 13, 2014 (GB) 1414337.4

(51) Int. Cl. A61F 2/34; A61F 2/30

(52) U.S. Cl. CPC A61F 2/34; A61F 2/30771; A61F 2002/3401; A61F 2002/3412; A61F 2002/3429; A61F 2002/30331; A61F 2002/30462; A61F 2002/30649; A61F 2002/3069; A61F 2002/3093; A61F 2002/3092

(57) ABSTRACT

An acetabular cup prosthesis comprises at least one recess in an edge of the cup and at least one attachment means. The attachment means has a projecting portion for attachment of the cup to an introducer (configured for insertion of the cup into a patient) and a retaining portion for retaining the attachment means on the cup. The retaining portion is located in a respective one of the recesses and is fixedly secured in the respective recess. A method of manufacture of the cup is also provided.

### RELATED APPLICATION

This application claims priority to United Kingdom Patent Application No. 1414337.4, filed on Aug. 13, 2014, the entire disclosure of which is incorporated by reference herein.

### FIELD OF THE INVENTION

This invention relates to an acetabular cup prosthesis. Particularly, but not exclusively, the invention relates to a metal or ceramic acetabular cup prosthesis for use in hip resurfacing.

### BACKGROUND TO THE INVENTION

Hip resurfacing is commonly performed where a patient's acetabulum is no longer functional. This involves removing and reshaping the acetabulum to allow a new, prosthetic acetabular cup to be fixed into the hip. The femoral head is also commonly replaced in order to provide both sides of a replaced joint. Different materials have been used for this, including metal, ceramic, polymer, and composite materials, which generally include a combination of a metal outer shell and polymer inner liner.

Commonly, acetabular cups are configured for press-fit fixation (e.g. by forcing a 50 mm outer diameter component into a 48 mm diameter hole). This can result in considerable deformation (e.g. in the range of 100 microns to over 350 microns), even where thick metal shells are employed, and so there is a risk that the cup will grip the femoral component leading to early acetabular component breakout. Alternative fixation features, such as large projecting pegs, are therefore sometimes employed. To aid fixation of these components, the external surface of the cup is often provided with a porous coating (e.g. by plasma spraying titanium particles) to encourage bone in-growth. However, such a coating only tends to provide limited contact between the bone and the cup leading to poor grip. In addition, the titanium particles can easily become dislodged, such that they then serve as abrasive debris.

It is important that the prosthesis be positioned highly accurately within the patient to ensure that the joint functions correctly in situ. An introducer is generally used to grip the acetabular cup as it is inserted into the patient and which allows the cup to be easily maneuvered into the correct position. There is therefore a need for an effective means of attaching the cup to the introducer.

It is an aim of the present invention to provide an improved acetabular cup prosthesis, designed with the above considerations in mind.

### SUMMARY OF THE INVENTION

According to a first aspect of the present invention there is provided an acetabular cup prosthesis comprising:
at least one recess in an edge of the cup; and
at least one attachment means having a projecting portion for attachment of the cup to an introducer, configured for insertion of the cup into a patient, and a retaining portion for retaining the attachment means on the cup, the retaining portion being located in a respective one of said recesses and fixedly secured in said respective recess.

According to a second aspect of the present invention there is provided a method of manufacture of an acetabular cup prosthesis, the method comprising:
forming an acetabular cup prosthesis with at least one recess in an edge of the cup;
providing at least one attachment means having a projecting portion for attachment of the cup to an introducer, configured for insertion of the cup into a patient, and a retaining portion for retaining the attachment means on the cup;
locating the retaining portion in a respective one of said recesses in the edge of the cup; and
fixedly securing the retaining portion in said respective recess.

Embodiments of the present invention therefore provide an acetabular cup prosthesis which can be produced in a variety of materials (e.g. metal, ceramic or polymer) and which is provided with an integral attachment means to aid handling of the cup and to facilitate accurate insertion of the cup into a patient. Notably, the attachment means is fixedly secured to (i.e. locked onto) the cup such that the retaining portion is non-slideable and non-removable from the recess. This provides greater control and increased accuracy when placing and orientating the cup in a patient.

It will be understood that, after the cup has been correctly positioned in the patient, the projecting portion of the attachment means will be removed from the cup but the retaining portion will remain in the cup recess. This is advantageous in that the recess is effectively filled with and sealed by the presence of the retaining portion to prevent debris and/or fluids from accumulating in the recess and potentially damaging the cup, femoral head or acetabulum in use.

It will be noted that the attachment means is fixedly secured within the recess of the cup as part of the manufacturing process for forming the prosthesis. Accordingly, an advantage of the present invention is that attachment means is readily available for the surgeon to handle and orientate the cup, without having to (correctly) attach any additional components first. In addition, the fact that the attachment means projects from the edge means that it does not project from the curved outer surface of the metal shell. Accordingly, the attachment means does not interfere with the placement of the cup in the prepared bone of the acetabulum. In other words, the acetabulum can be prepared as normal, simply taking into account the size and shape of the outer shell of the cup. No additional cut-outs are required in order to accommodate the attachment means.

The projecting portion may be frangible, cuttable or meltable in a region adjacent to the edge of the cup so that the projecting portion can be removed after the cup has been placed in the patient.

The attachment means may be fixed within the recess by moulding (e.g. injection moulding or compression moulding), hot-forming and/or by mechanical means.

The retaining portion may comprise an enlarged portion relative to an opening of the recess in the edge of the cup such that the enlarged portion constitutes mechanical means for fixing the attachment means to the cup. The enlarged portion may be formed by forming or affixing a mass (e.g. a ball or nut) to an end of the retaining portion. Alternatively, the enlarged portion may be formed by tying a knot in an end of the retaining portion. For example, the attachment means may comprise a polymer cable and a knot may be tied at one end of the cable to form the enlarged portion.

In one embodiment, the attachment means may comprise a metal cable and the enlarged portion may be constituted by a metal ball which is crimped or welded to the metal cable.

In another embodiment, the attachment means may comprise a polymer cable and the enlarged portion may be constituted by a polymer mass which is moulded to an end of the polymer cable. In particular embodiments, the polymer cable may be braided and the end of the polymer cable may be unravelled prior to moulding the polymer mass to the end of the polymer cable. Accordingly, the polymer mass will be able to get between individual fibres of the polymer cable when moulding onto it, in order to provide chemical bonds with the polymer cable over a larger surface area. It is believed that such an approach will help to strengthen the attachment means whilst fixing it to the cup.

The cup may comprise an undercut to accommodate the enlarged portion and retain the attachment means on the cup. The undercut may be located a distance away from the edge of the cup (e.g. deep within the body of the cup) so as to transfer stresses transmitted through the attachment means to the cup, from a thin and more fragile edge of the cup to a thicker, more robust region of the cup.

The undercut may be formed by machining or by using a lost-wax method or similar technique (e.g. using a leachable ceramic).

In particular embodiments, a locking element may be provided in the recess for the attachment means to lock onto. The locking element may therefore help to prevent the retaining means from inadvertently being pulled out of the cup. The locking element may comprise a post (e.g. stud) which the retaining portion is fixed to. The post may be provided centrally within the undercut portion of the recess. In some embodiments, a ceramic piece may be employed to define the shape of the recess and/or locking element during manufacture of the cup (e.g. when the cup is formed from metal). Accordingly, the ceramic piece may be incorporated into a metal casting and then leached out to form the recess in the cup edge. A polymer attachment means may then be fixed in the recess by injection moulding, compression moulding or hot-forming.

In some embodiments, the undercut may be formed by providing a second recess (exterior recess) which extends from an exterior (convex) surface of the cup into the recess in the edge of the cup (edge recess). The second recess may be larger in diameter than the recess in the edge of the cup. Advantageously, the second recess can provide access to an end of the attachment means for forming the enlarged (retaining) portion.

Optionally, the second recess may be sealed (e.g. by over-moulding) after the enlarged portion has been formed and located therein. Accordingly, a seal may be provided in the second recess after the retaining portion has been located and fixed therein. This is advantageous in preventing debris from passing through the edge recess and forming osteolysis in bone behind the cup. The seal may comprise polymer (e.g. polyester, polyethylene, UHMWPE, low molecular weight polyethylene, PEEK or nylon) and may be injection moulded, compression moulded or hot-formed. The second recess may comprise a second undercut to retain the seal therein. It will be understood that the second undercut may not be necessary when the cup itself is formed of polymer since the seal may be chemically bonded to the cup.

Some further advantages of sealing the second recess are listed below:

1) If using a polymer attachment means in a polymer cup then over-moulding the end of the attachment means in the recess will chemically link all three components together.

2) If using a non-polymer braided attachment means, a polymer seal will mechanically lock into all recesses provided by the external surface of the braided attachment means.

3) When a knot is tied in the end of the retaining portion, the polymer seal will prevent the knot from untying under high tension when in use.

4) Although unlikely, a knot in the retaining portion could possibly work its way out of the recess into the bone-implant interface, when in use, but the seal will prevent this from happening.

5) When using a metal attachment means, the end of the retaining portion may be sharp but the seal will cover over any sharp portions and prevent them from causing damage.

6) The seal can provide an external surface which can be coated to promote bone ingrowth. For example, the seal - along with the rest of the external surface of the cup—could be coated with a porous particle surface (e.g. by spraying hydroxyapatite, with or without titanium, or by using ‘cold’ plasma spraying of titanium).

In certain embodiments, the edge recess and the second recess may intersect to form a hole with a cross-shaped cross-section. In which case, the second recess may constitute the undercut in relation to the edge recess and the edge recess may constitute the second undercut in relation to the second recess such that no further undercuts are required.

The attachment means may comprise a cable, strap, rod, finger or wire. In particular embodiments, the attachment means may comprise a mono-filament element. However, it may be preferable for the attachment means to comprise a poly-filament element (e.g. a mechanically braided element) since such structures exhibit better strength properties when in tension.

The attachment means may be formed from any suitable materials and may comprise a polymer (e.g. polyester, polyethylene, UHMWPE, low molecular weight polyethylene, PEEK or nylon) or metal. The attachment means could be fibre reinforced to improve tension strength and minimise the risk of breaking.

The cup may comprise polymer, metal or ceramic and may include a porous convex surface for bone ingrowth. The metal may comprise, for example, titanium, cobalt, chromium, zirconium or zirconium-oxide or any combination thereof. For example, the cup may comprise a ceramicised metal (e.g. having an oxidised surface zirconium alloy). The cup may be partly or completely formed from metal. It may be formed in one piece. Alternatively, the cup may be at least partly or completely formed from ceramic material. Again, the cup may be formed in one piece. In other embodiments, the cup may be partly or completely formed from polymer material and may be formed in one piece.

Where the cup is formed only of metal or only of ceramic material, the cup is essentially a one-piece prosthesis with no separate inner liner forming the bearing surface; the bearing surface is formed from the cup material itself. With such a one-piece prosthesis, accurate positioning of the cup can be difficult, as the inner bearing surface is, by design, smooth; projections and locating elements formed from metal and ceramic can interfere with the movement of the cup when implanted into a patient. Provision of attachment means on the prosthesis and an introducer to insert the cup into a patient allows the cup to be accurately located. Further, provision of relatively soft attachment means, rather than forming them from the body of metal or ceramic, allows for them to be cut once the introduction of the cup is complete and it is correctly seated in the acetabulum. This then removes projections which could otherwise interfere with the full movement of a femoral head within the cup, and which could otherwise cause wear or failure of the cup or joint.

In this way, while the prosthesis can easily be correctly and accurately positioned, once the positioning is complete, the attachment means can be cut, above and adjacent to the edge of the cup, thus removing the protruding part of the attachment means, and reducing the likelihood of interference between the attachment means and the joint after insertion.

A cross-section of an opening of the recess at the edge is preferably smaller than a (parallel) cross-section of the recess below the edge to form an undercut. This allows the attachment means to be held securely within the recess. The cross-sectional size of the recess may increase in a direction away from the opening, into the cup body. This increase in cross-sectional size may occur gradually, e.g. linearly, or may increase sharply, for example to form a lip or flange in the recess at the opening, behind which (i.e. further into the recess from the opening) the cross-sectional size of the recess is larger. The shape of the recess and/or undercut is not limited but could be substantially cylindrical, spherical, square, triangular, conical, pyramidal, toroidal, circular, disc-shaped, cross-shaped or inverted T-shaped. It will be understood that, due to the relatively thin nature of the edge of the cup, the recess and/or undercut may extend a greater distance in a direction generally parallel to the edge of the cup than in a direction generally transverse to the edge of the cup.

In certain embodiments, the projecting portion of the attachment means may be sloped or curved inwardly towards the centre of the cup. For example, the projecting portion may project from the edge at an angle of, say, 5, 10, or 20 degrees inwardly from the perpendicular direction.

The attachment means may comprise one or more loops having a first end and a second end secured, respectively, within first and second recesses in the cup edge; the first and second ends constituting first and second retaining portions. In one embodiment, two loops are provided, one on each side of the cup. The loops may be injection moulded into the recesses in the cup. After insertion of the cup into a patient, the loops may be removed (e.g. by cutting above the first and second ends, adjacent the opening of the recess) so as to leave the cup with a relatively flush, smooth surface.

An aperture may be provided at a free end of the projecting portion; the aperture being configured for attachment to a projection on an introducer.

In particular embodiments, the attachment means may comprise one or more projecting portions having a serrated surface. Alternatively, the attachment means may comprise one or more projecting portions having a device configured to lock onto a serrated surface. The device may include an opening having a ridge arranged to locate between two adjacent serrations. The device may be configured to easily accept the serrated surface (i.e. to allow the serrated surface to be inserted into it) but to prevent the serrated surface from being removed from its grasp. In other words, the device may be configured to allow the serrated surface to be passed through it in one direction but to prevent the serrated surface from passing through it in the opposite direction. Thus, the attachment means in these embodiments may take the form of cable ties, with either the serrated surface or the device for locking onto the serrated surface being provided as the attachment means on the cup and the other of the serrated surface or the device for locking onto the serrated surface being provided on the introducer.

In some embodiments, the projecting portion may have a smooth exterior surface. In which case, the introducer may comprise gripping means for gripping onto the projecting portion. The gripping means may comprise teeth arranged to bite into the projecting portion.

In other embodiments, the projecting portion may be provided with an enlarged portion at its free end, configured for gripping by an introducer. The enlarged portion may be spherical. In a certain embodiment the enlarged portion may be generally conical and orientated with its tip at the free end of the projecting portion. In one embodiment the enlarged portion may comprise two or more conical portions stacked with their tips all towards the free end of the projecting portion.

The projecting portion may include a neck configured such that rotation of the projecting portion with respect to the cup will cause the projecting portion to shear at the neck. The neck may be located adjacent the edge of the cup.

### BRIEF DESCRIPTION OF EMBODIMENTS OF THE INVENTION

Detailed embodiments of the invention will now be described, purely by way of example, with reference to the accompanying drawings, in which:

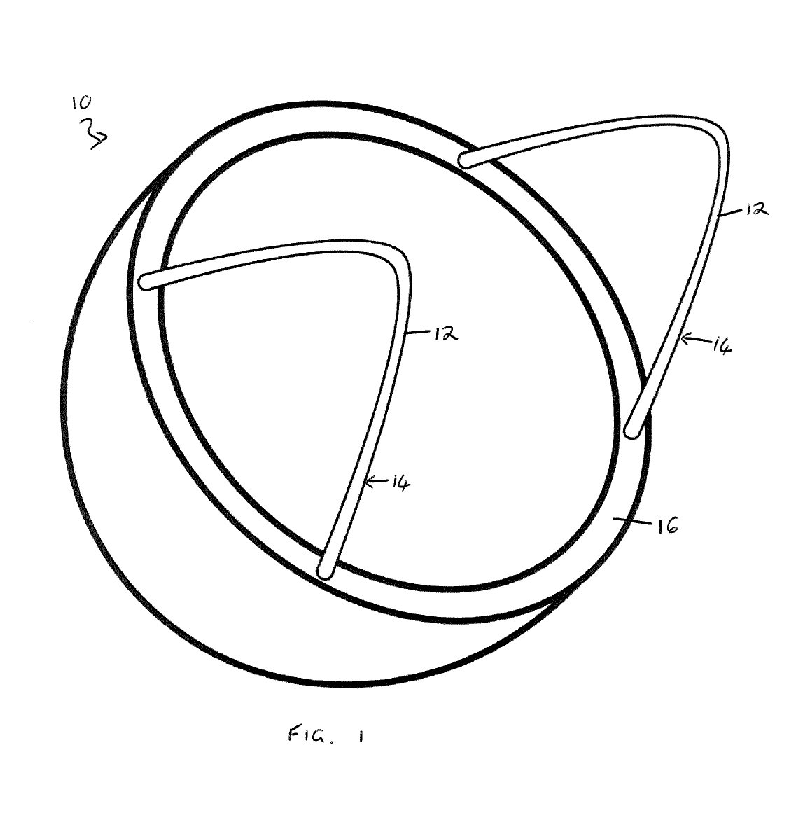

FIG. 1 shows an acetabular cup according to a first embodiment of the invention;

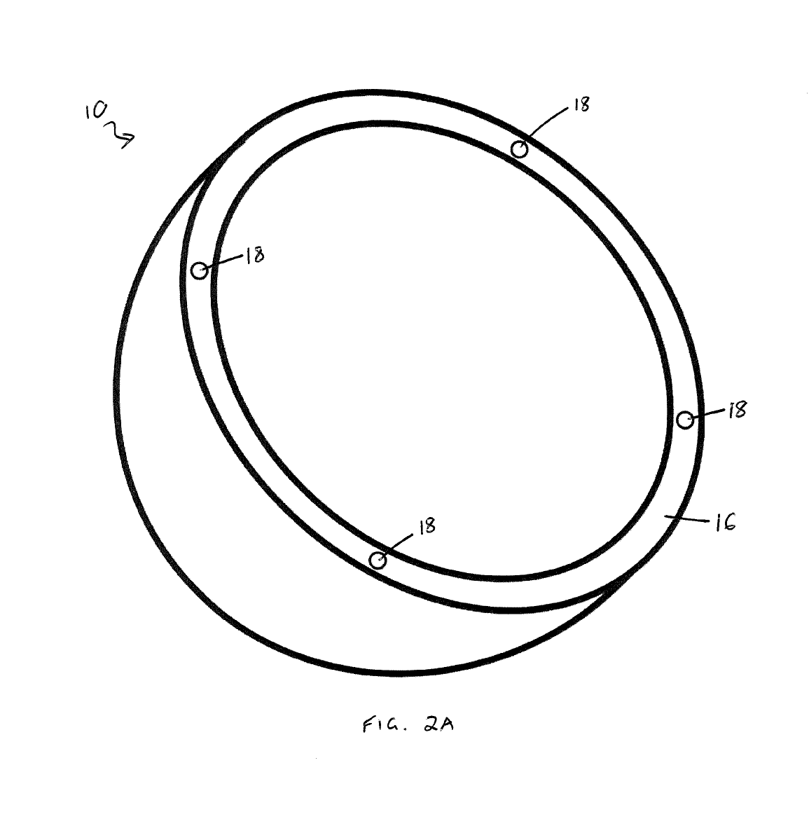

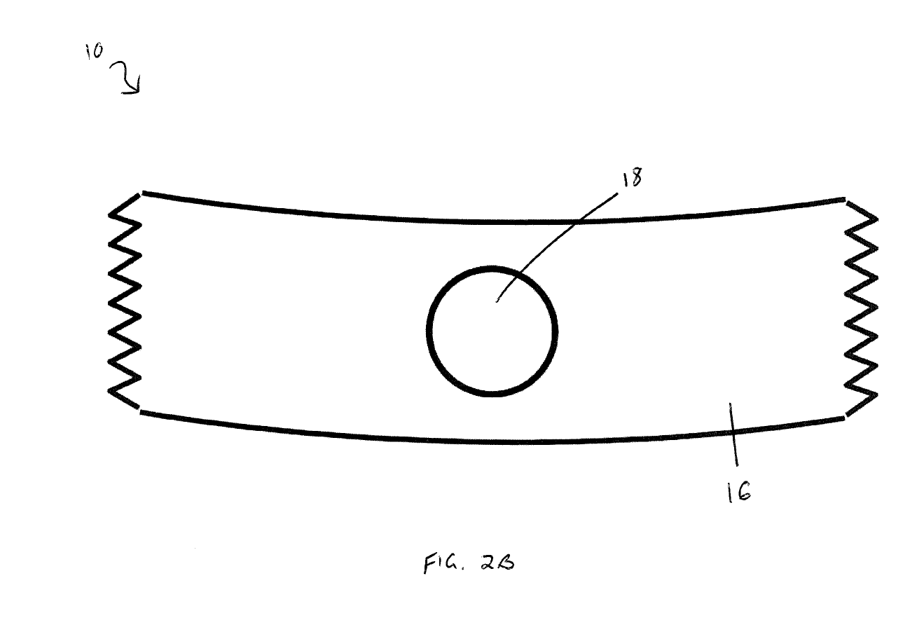

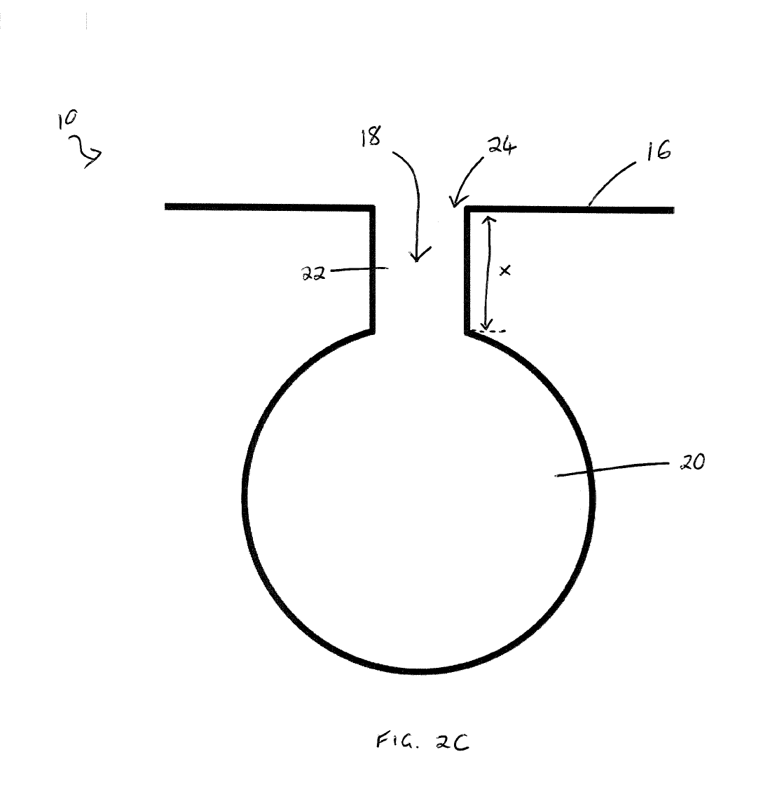

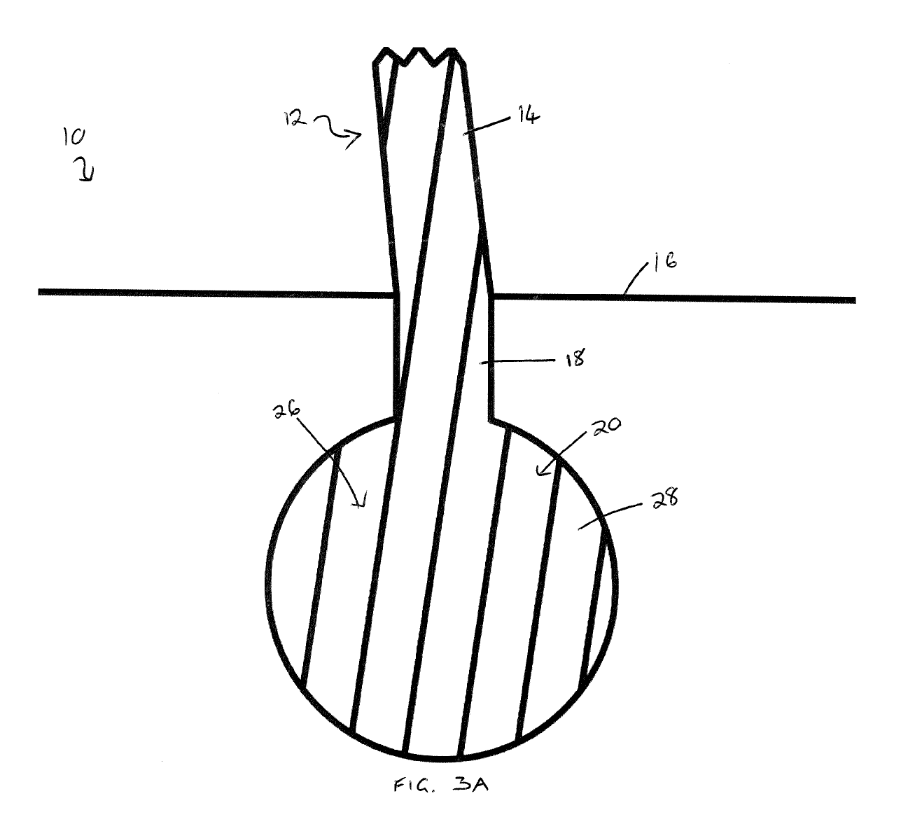

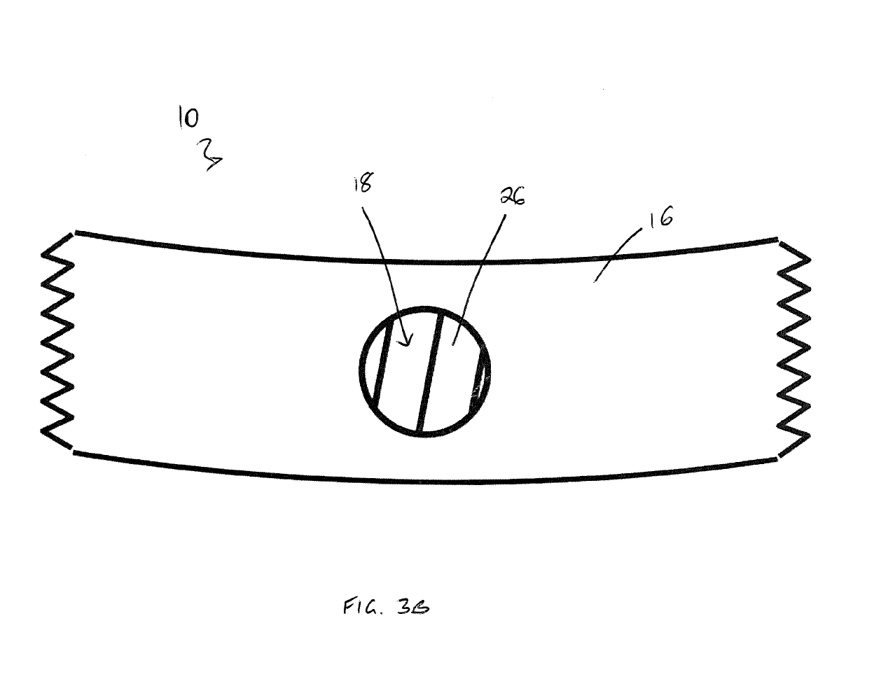

FIG. 2A shows the cup of FIG. 1 prior to attachment of the attachment means;

FIG. 2B shows an enlarged partial view of an edge of the cup of FIG. 2 showing one recess;

FIG. 2C shows a longitudinal cross-section through the recess of FIG. 2B, prior to attachment of the attachment means;

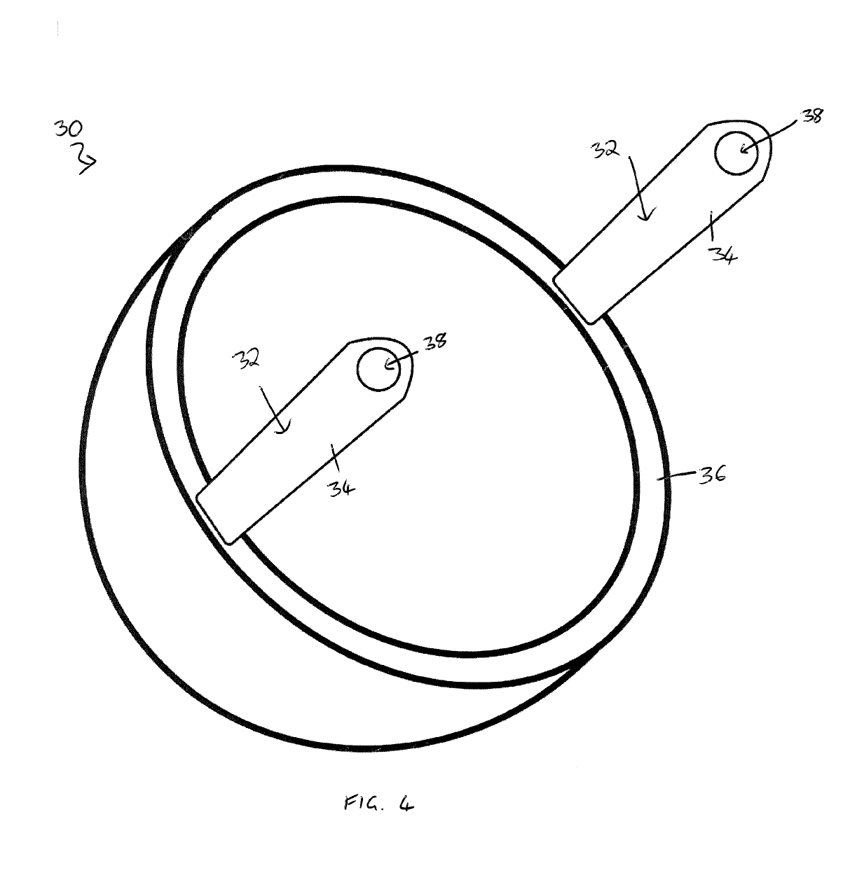

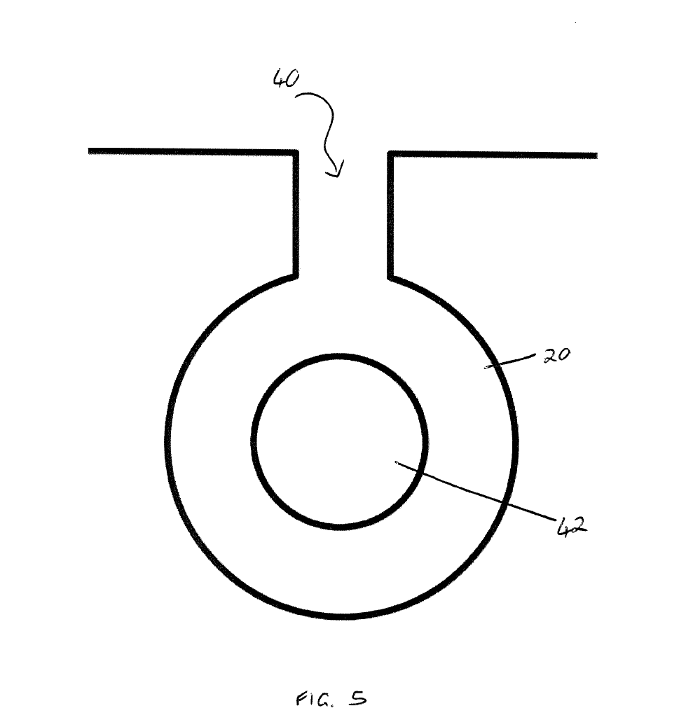

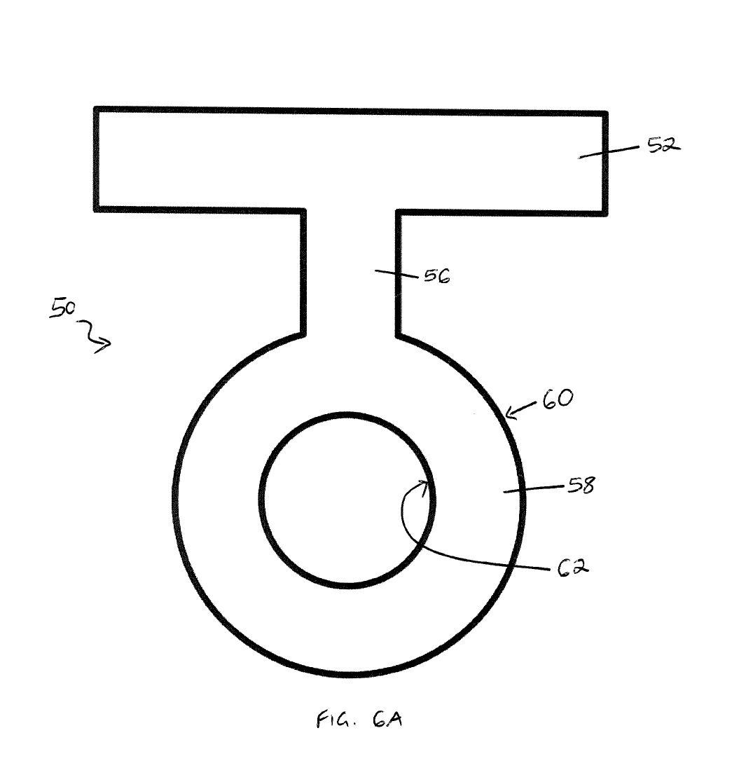

FIG. 3A shows a view similar to that of FIG. 2C, after attachment of the attachment means;

FIG. 3B shows a view similar to that of FIG. 2B after the projecting portion of the attachment means has been removed;

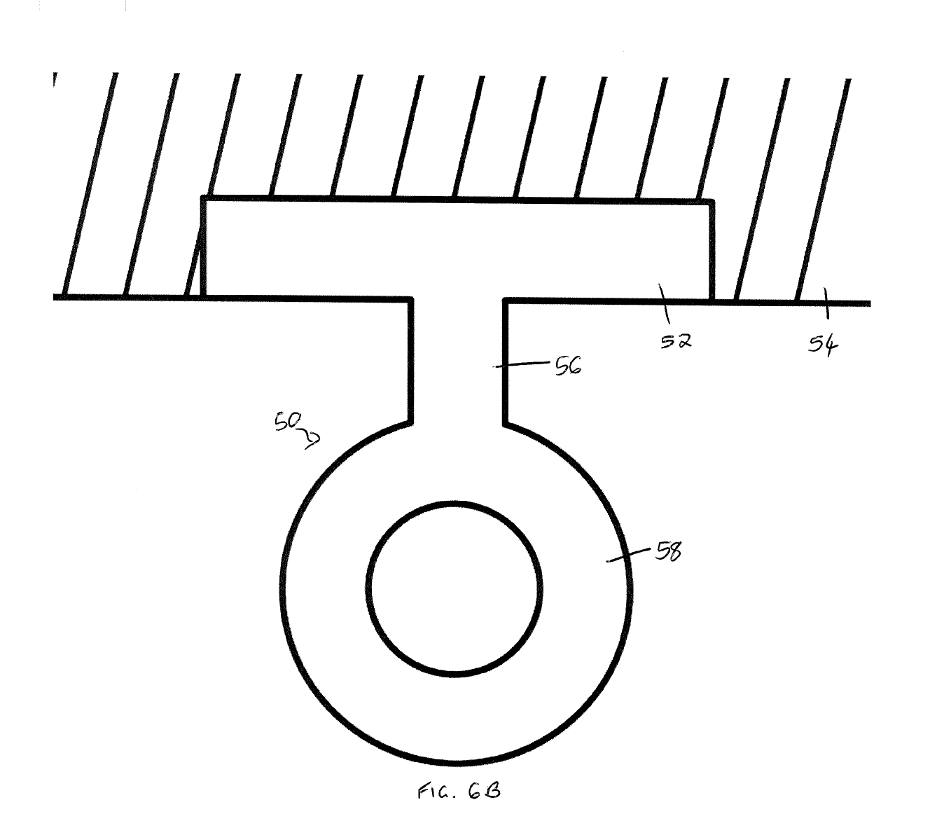

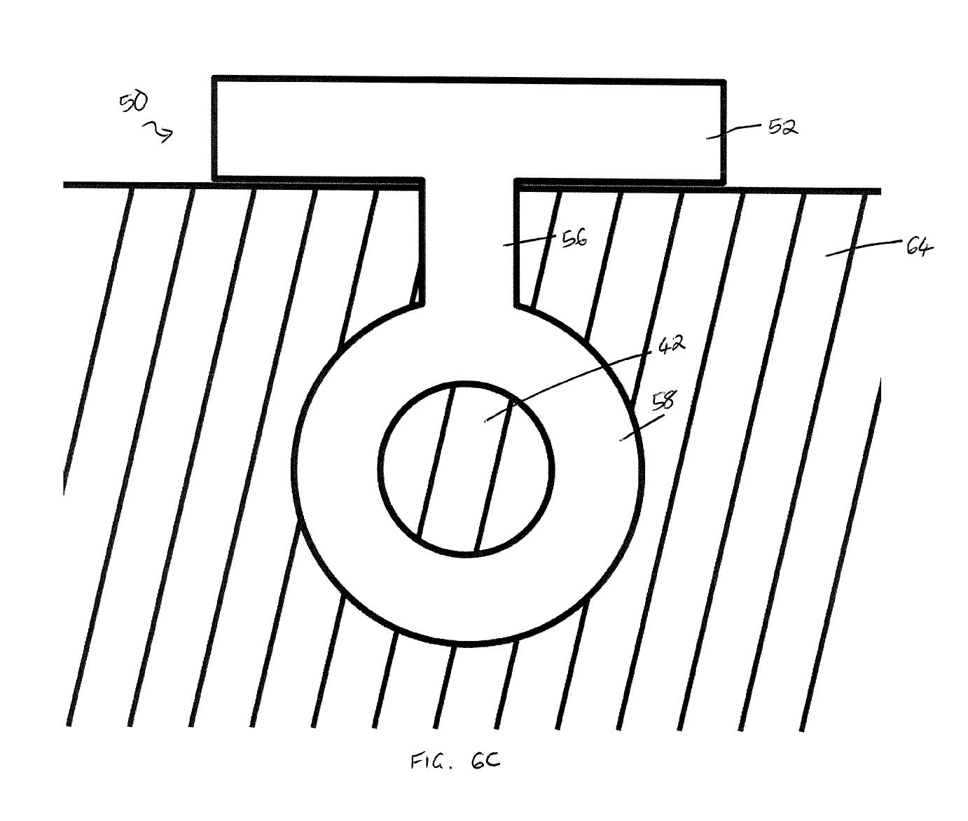

FIG. 4 shows an acetabular cup according to a second embodiment of the invention;

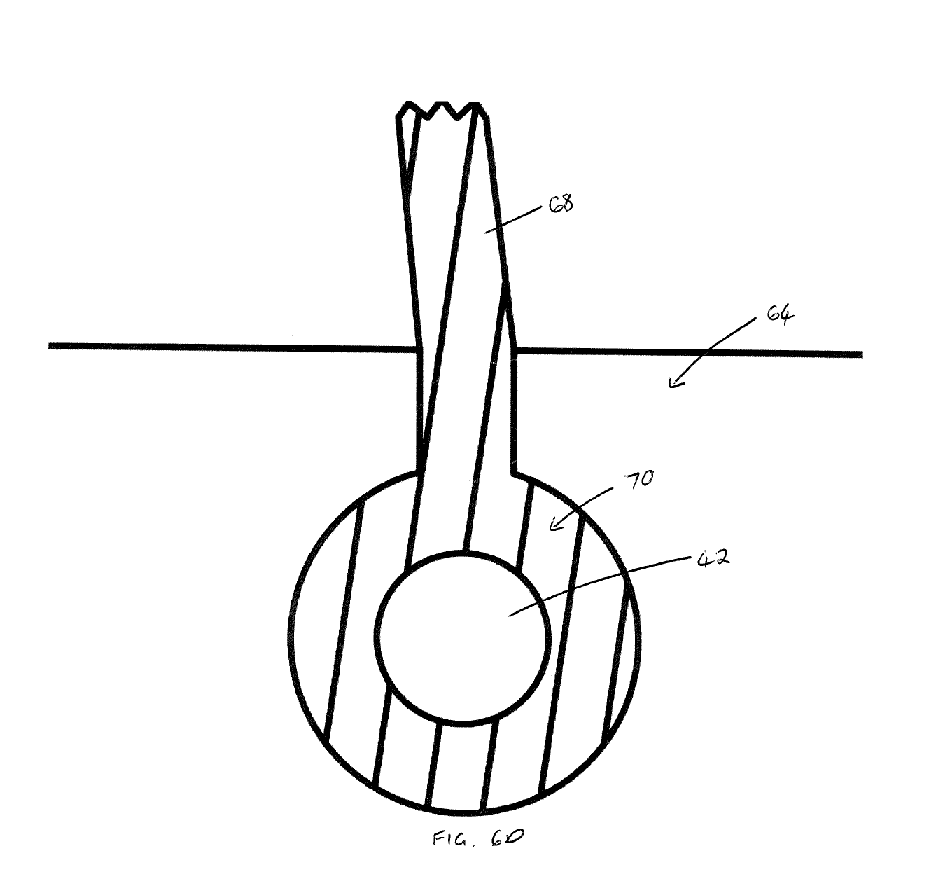

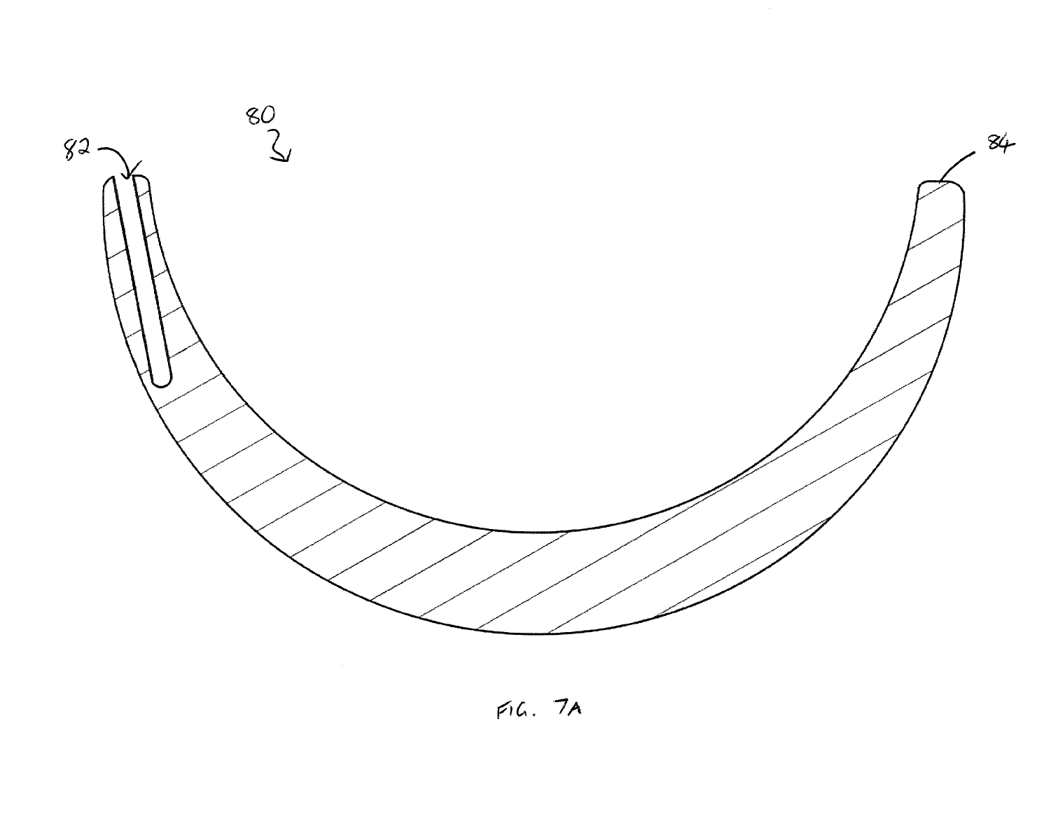

FIG. 5 shows a view similar to that of FIG. 2C but wherein a locking stud is provided in the recess;

FIGS. 6A through 6D show a first method of manufacturing a cup with a locking stud in accordance with FIG. 5;

More specifically, FIG. 6A shows a side view of a leachable ceramic core used to define the shape of the recess and locking stud in the cup;

FIG. 6B shows a side cross-sectional view of the ceramic core of FIG. 6A when fixed in an investment mould ready for molten metal to be poured into the mould;

FIG. 6C shows a side cross-sectional view of the ceramic core of FIG. 6A when disposed in a moulded metal cup;

FIG. 6D shows a side cross-sectional view after the ceramic core has been leach away and an attachment means has been moulded in the recess;

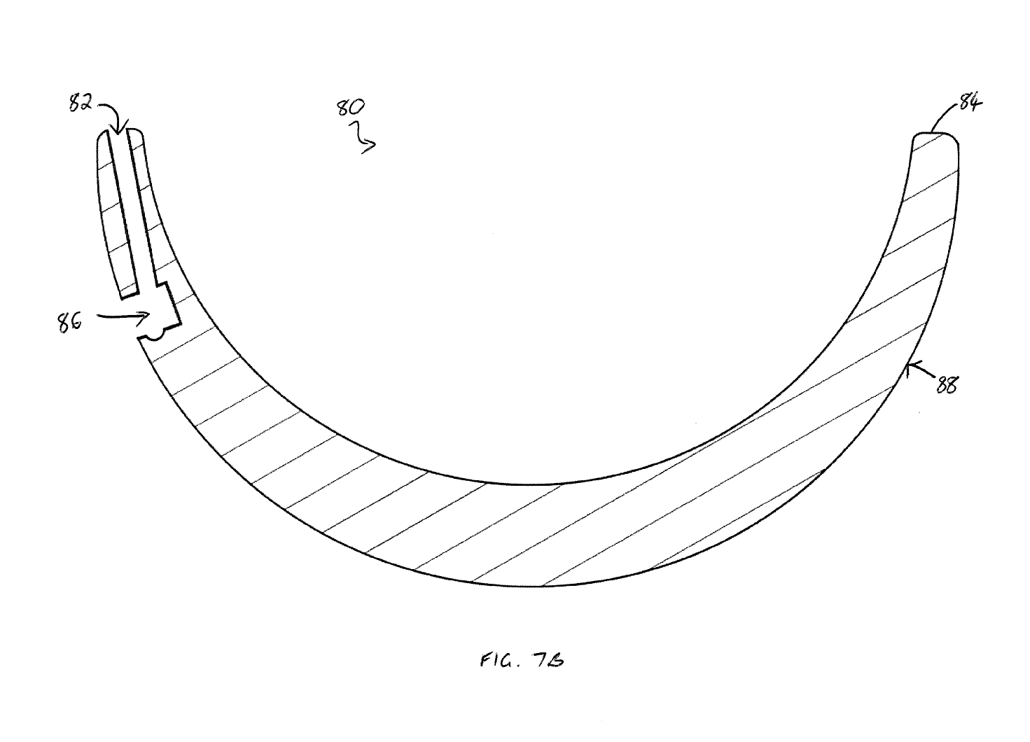

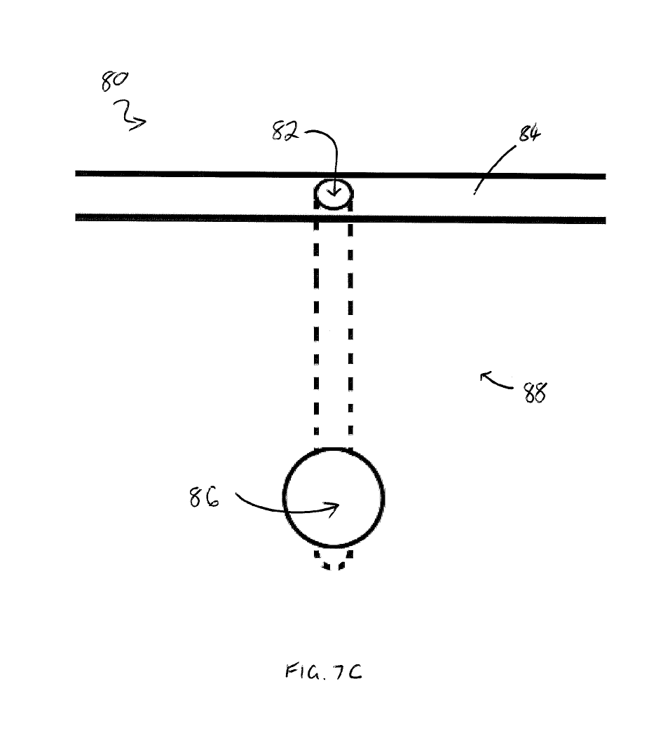

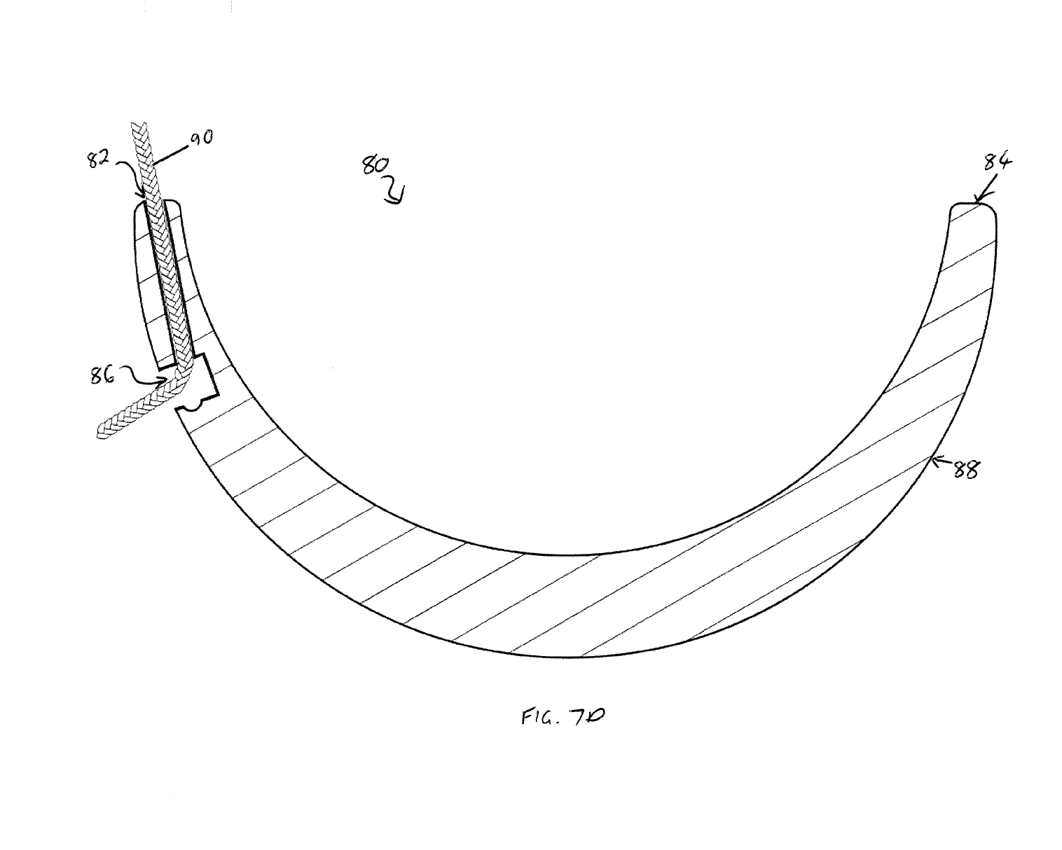

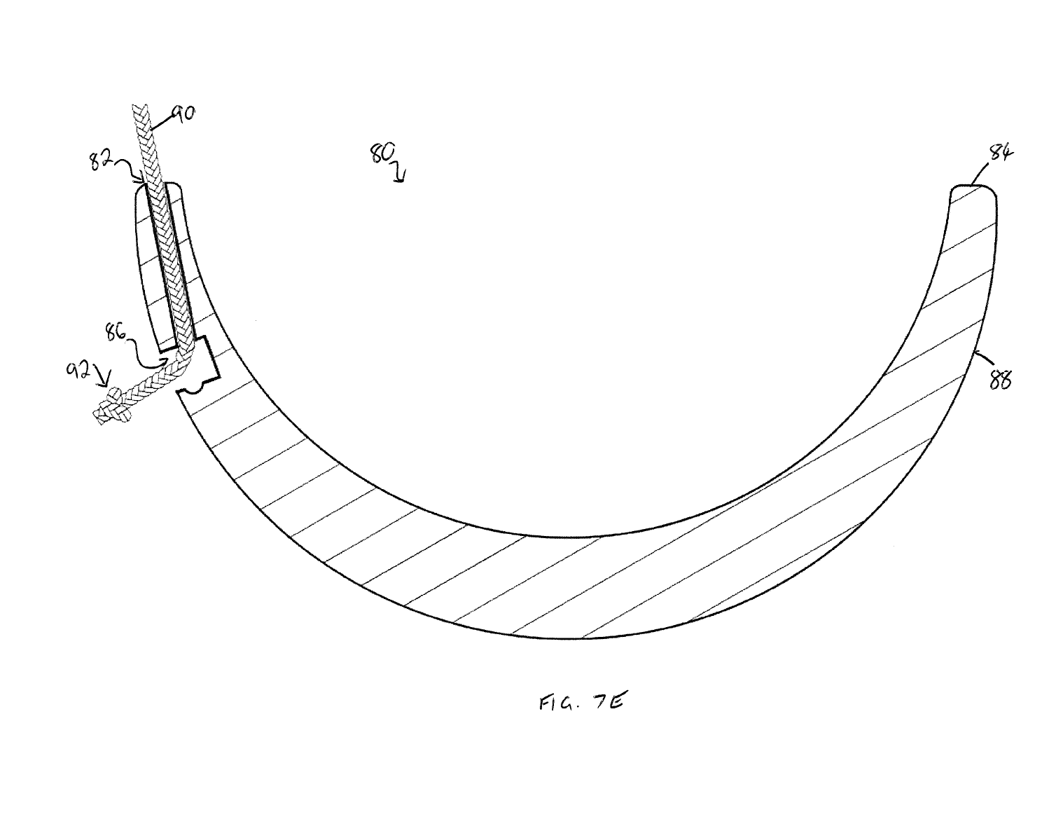

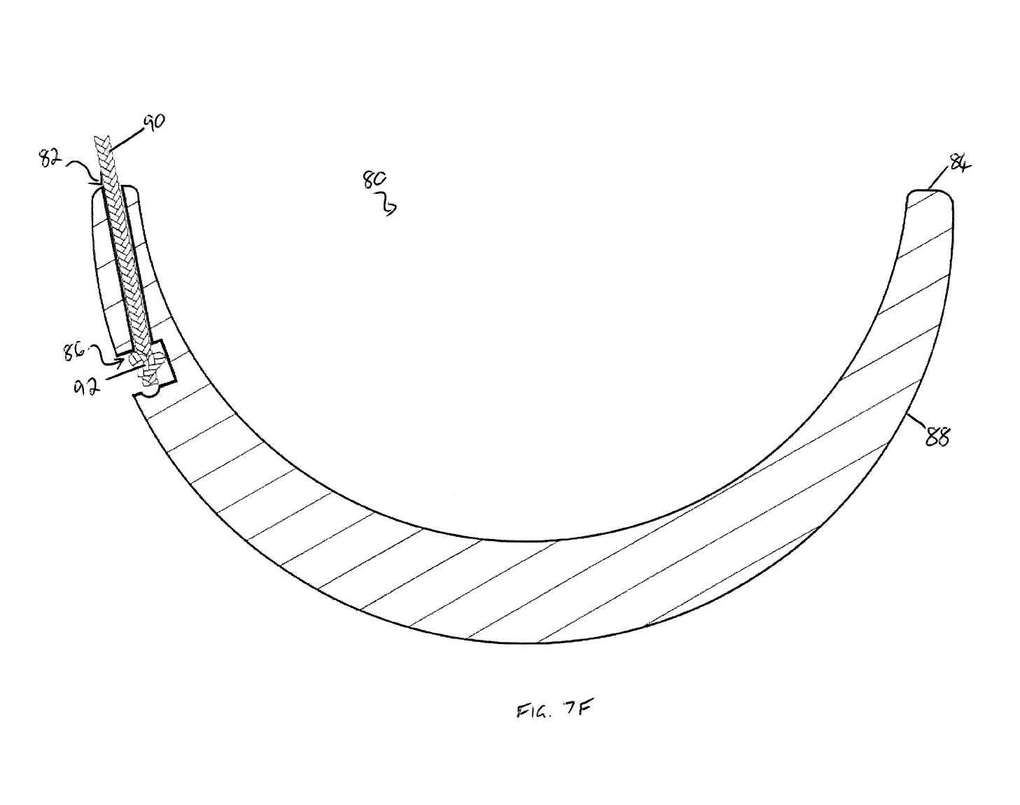

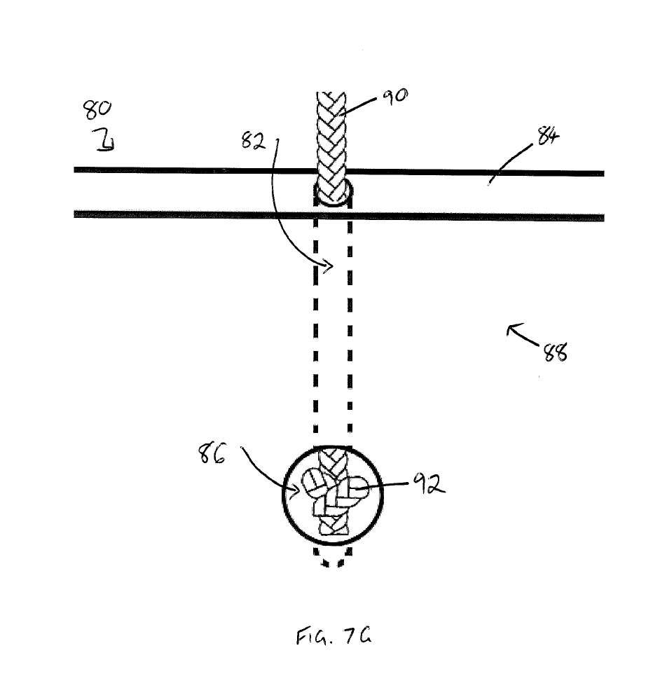

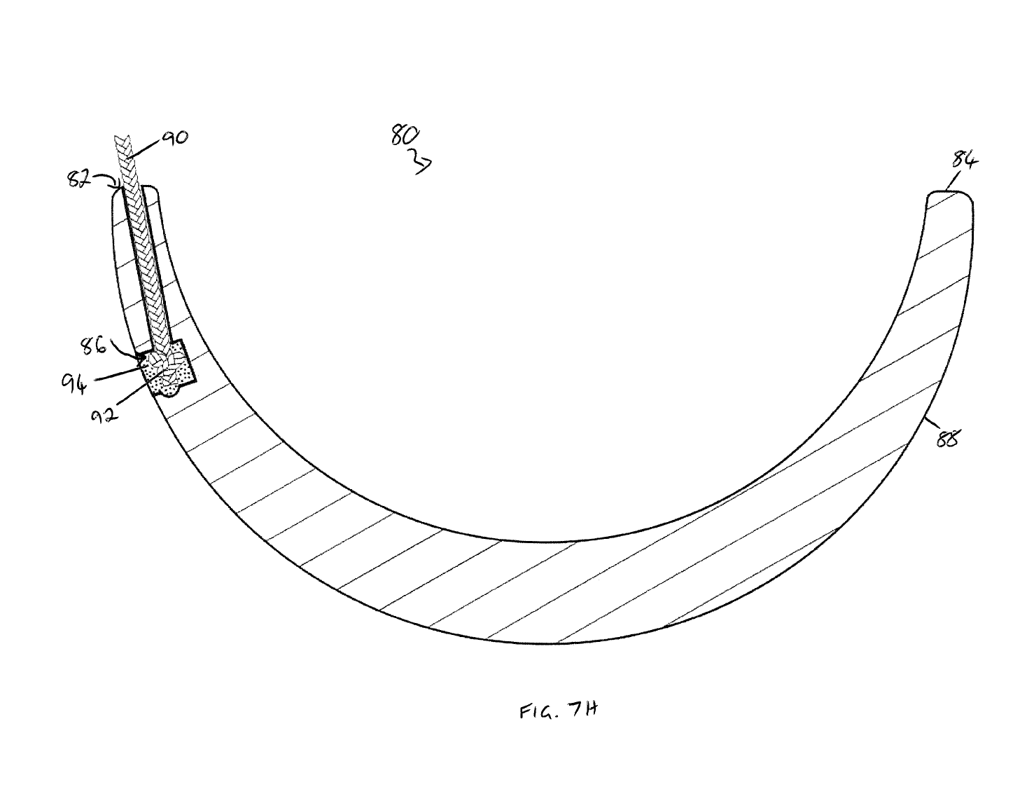

FIGS. 7A through 7I show a second method of manufacturing a cup in accordance with an embodiment of the invention;

More specifically, FIG. 7A shows a side cross-sectional view of a cup after a first recess has been provided in the edge of the cup;

FIG. 7B shows the cup of FIG. 7A after a second recess has been provided in the exterior surface of the cup, the second recess passing through the first recess;

FIG. 7C shows a side perspective view of the cup of FIG. 7B, illustrating the first and second recesses;

FIG. 7D shows the cup of FIG. 7B after an end of a braided polymer attachment means has been passed through the first recess and out of the second recess;

FIG. 7E shows the cup of FIG. 7D after a knot has been tied in the end of the attachment means;

FIG. 7F shows the cup of FIG. 7F after the attachment means has been pulled so that the knot is located in the second recess;

FIG. 7G shows a side perspective view of the cup of FIG. 7F, showing the knot in the second recess;

FIG. 7H shows the cup of FIG. 7F after a seal has been provided around the knot in the second recess;

FIG. 7I shows a side perspective view of the cup of FIG. 7H, showing the seal in the second recess;

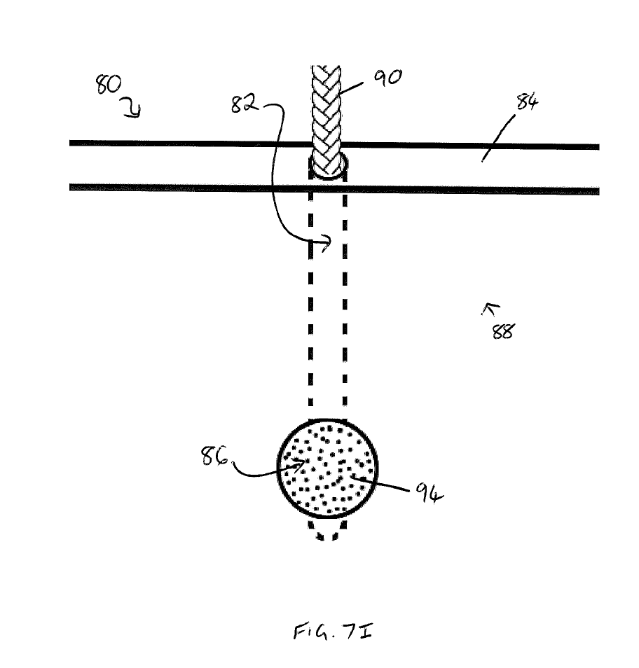

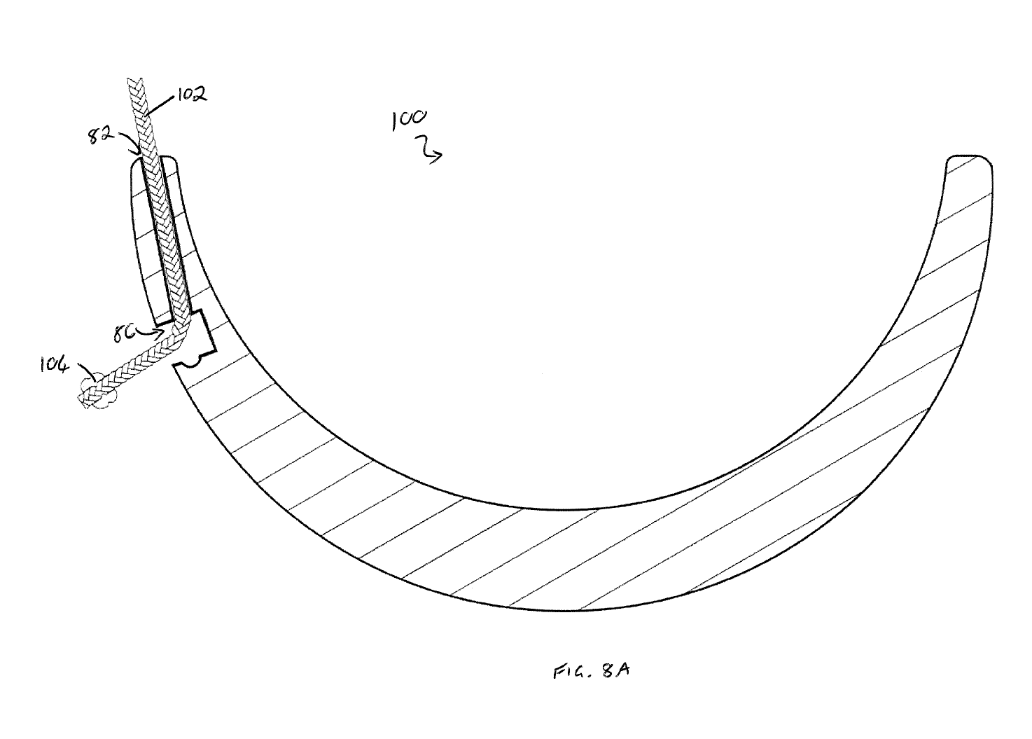

FIGS. 8A through 8C show a third method of manufacturing a cup in accordance with an embodiment of the invention;

More specifically, FIG. 8A shows a view similar to that of FIG. 7E but wherein a mass (rather than a knot) is provided on the end of the attachment means;

FIG. 8B shows the cup of FIG. 8A after the attachment means has been pulled so that the mass is located in the second recess;

FIG. 8C shows the cup of FIG. 8B after a seal has been provided around the mass in the second recess;

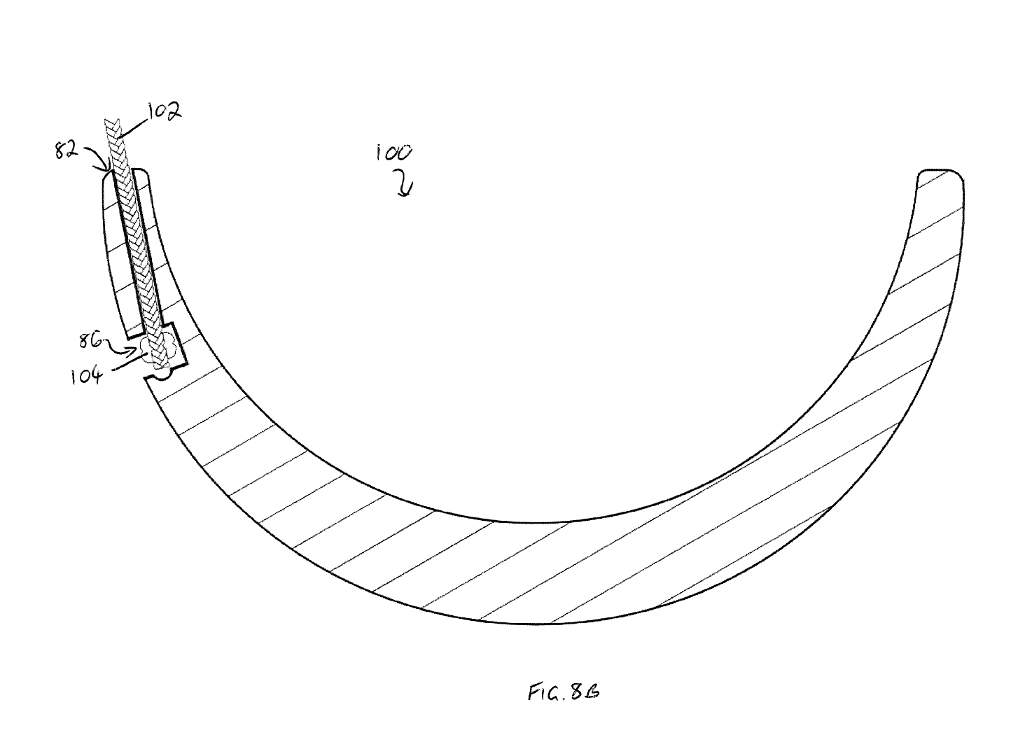

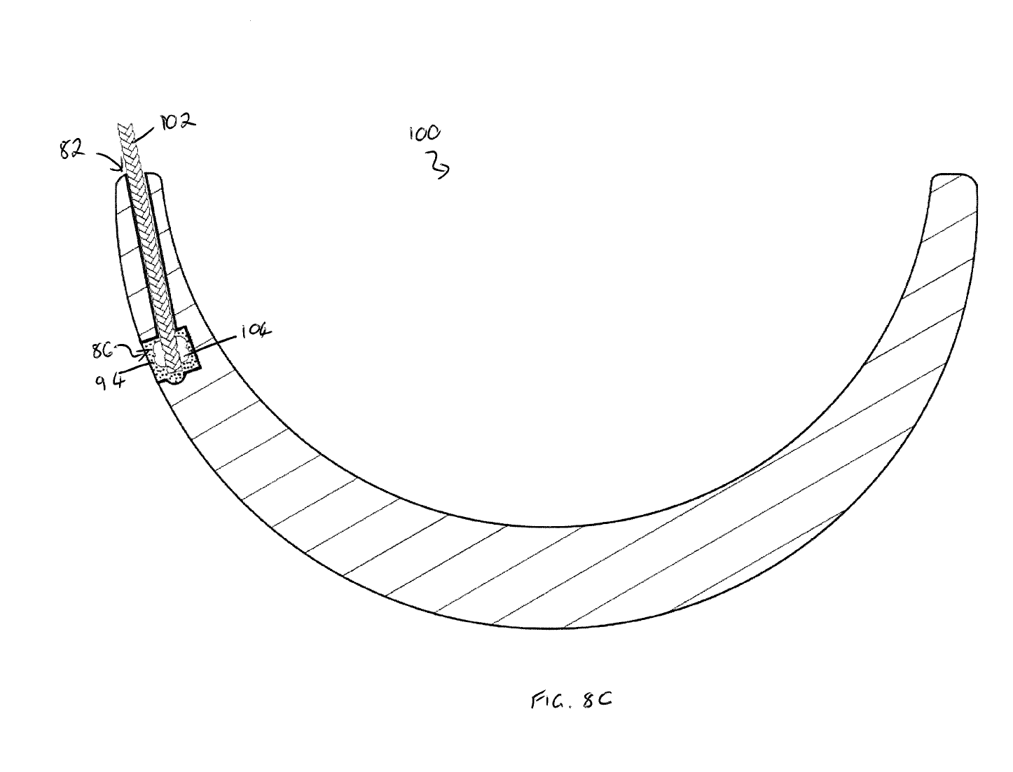

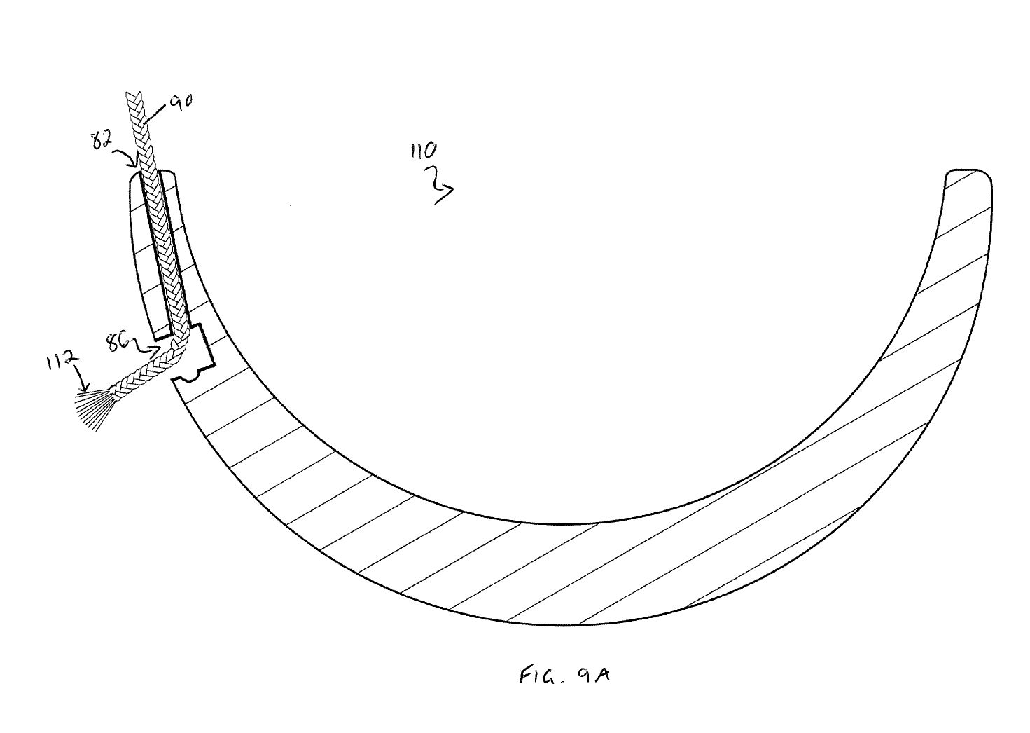

FIGS. 9A through 9C show a fourth method of manufacturing a cup in accordance with an embodiment of the invention;

More specifically, FIG. 9A shows a view similar to that of FIG. 7E but wherein the end of the attachment means has been frayed;

FIG. 9B shows the cup of FIG. 9A after the attachment means has been pulled so that the frayed end is located in the second recess; and

FIG. 9C shows the cup of FIG. 9B after a seal has been provided around the mass in the second recess.

### DETAILED DESCRIPTION OF EMBODIMENTS OF THE INVENTION

FIG. 1 shows an acetabular cup prosthesis 10 according to a first embodiment of the invention. As shown, the cup 10 is provided with attachment means in the form of two loops 12. Each loop 12 comprises a projecting portion 14 for attachment of the cup 10 to an introducer configured for insertion of the cup into a patient (not shown), and two retaining portions (not shown in FIG. 1) for retaining the loops 12 on the cup 10. As will be described below, the retaining portions are located in recesses provided in an edge 16 of the cup 10 and are fixedly secured therein. In this particular embodiment, the cup 10 is formed of metal and the loops 12 are formed from 1.5 mm diameter UHMWPE braided cord.

FIG. 2A shows the cup 10 of FIG. 1 prior to attachment of the two loops 12. Accordingly, four circular recesses 18 can be seen in the edge the cup 10—one for receipt of each end of each of the loops 12. In this case, the opening of each recess 18 is 1.5 mm in diameter to accommodate the 1.5 mm diameter cord for the loops 12. In other embodiments, the recesses may have an opening which is non-circular (e.g. square or slot-shaped). FIG. 2B shows an enlarged partial view of the edge 16 of the cup 10 of FIG. 2A showing one recess 18.

FIG. 2C shows a longitudinal cross-section through the recess 18 of FIG. 2B, prior to attachment of the loops 12. Thus it can be seen that each recess 18 comprises an undercut 20 in the shape of an upstanding circular disc (of diameter 5 mm) which is located a distance X away from the edge 16. Preferably, X is at least 3 mm, more preferably at least 5 or 10 mm. A neck 22 (of length X) is provided between the undercut 20 and the opening 24 of the recess 18.

FIG. 3A shows the recess 18 after attachment of an end of one of the loops 12. The portion of the loop 12 above the edge 16 constitutes the projecting portion 14 of the attachment means and the portion of the loop 12 below the edge 16 constitutes the retaining portion 26 of the attachment means. In this case, the loop 12 has been injection moulded into the recess 18 such that the polymer of the loop 12 has formed an enlarged portion 28 which fills the undercut 20 to lock the attachment means to the cup 10. Once the attachment means has been used to securely attach the cup 10 to an introducer and the cup 10 has been correctly positioned in a patent, the projecting portions 14 of the loops 12 are cut close to the edge 16 and the retaining portions 26 remain in the recess 18, as shown in FIG. 3B, for the lifetime of the cup 10.

FIG. 4 shows an acetabular cup 30 according to a second embodiment of the invention in which two opposed polymer straps 32 are provided as attachment means.

Each strap 32 is formed from a flat piece of polyethylene approximately 1 mm thick and 50 mm long. As shown, each strap 32 has a projecting portion 34 projecting from an edge 36 of the cup 30. At a free end of each strap 32 there is a 5 mm diameter circular hole 38 configured for attachment to an introducer (not shown). Although not shown, the other end of each strap 32 forms a retaining portion which fixedly secures the strap 32 to the cup 30. The retaining portion may be similar to that shown in FIG. 3A or may be as described in an embodiment below.

FIG. 5 shows an alternative recess 40 which is similar to that shown in FIG. 2C but wherein a locking stud 42 is provided centrally within the undercut 20 to more securely fix a retaining portion to the cup. In this embodiment, a polymeric retaining portion may be injection moulded into the recess 40 and around the locking stud 42. Alternatively, an end of the strap 32 may be split into two halves and inserted into the recess 40 such that each half passes around either side of the locking stud 42 until they meet below the locking stud 42. The inserted end of the strap 32 would then be melted (e.g. by heating the cup itself) so as to melt fuse the two halves around the locking stud 42 to form the retaining portion of the attachment means.

FIGS. 6A through 6D show a first method of manufacturing a cup with a locking stud in accordance with FIG. 5. In this example, a leachable ceramic core 50 is employed to define the shape of the recess 40 and locking stud 42. The core 50 comprises a rectangular base 52 which is accommodated in an investment mould 54 (as shown in FIG. 6B), a neck 56 defining an opening channel for the recess 40 and a ring 58 which has an outer surface 60 defining the undercut 20 and an inner surface 62 defining the locking stud 42. FIG. 6C shows the core 50 disposed in a metal cup 64 after it has been cast in the mould 54. The core 50 is then leached out of the cup 64 to leave the required recess 40 and locking stud 42. It will be understood that the locking stud 42 is connected to the sidewalls of the cup 64 such that a circular channel 66 is provided around the locking stud 42. The recess 40 is then filled with the end of a polymer attachment means 68 (e.g. by injection moulding) to form a retaining portion 70 looped around the locking stud 42. The projecting portion of the attachment means 68 may be of any desired form, for example, loops as per FIG. 1 or straps as per FIG. 4.

FIGS. 7A through 7I show a second method of manufacturing a cup 80 in accordance with an embodiment of the invention. Again, the actual form of the projecting portion of the attachment means is not limited and could be as described in any of the above embodiments.

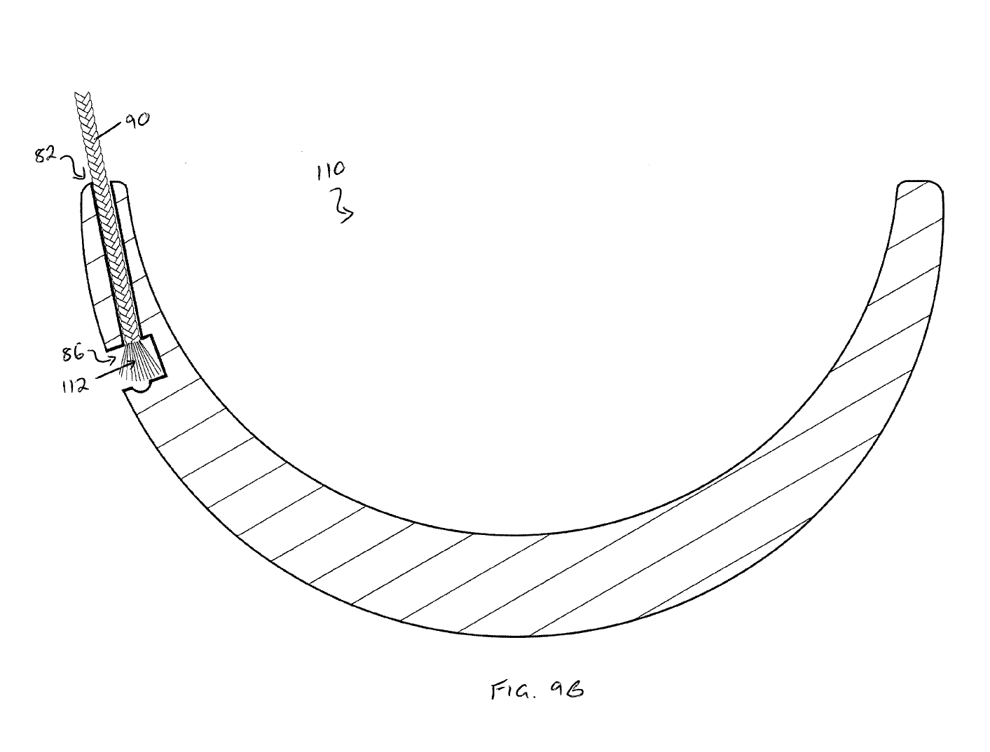

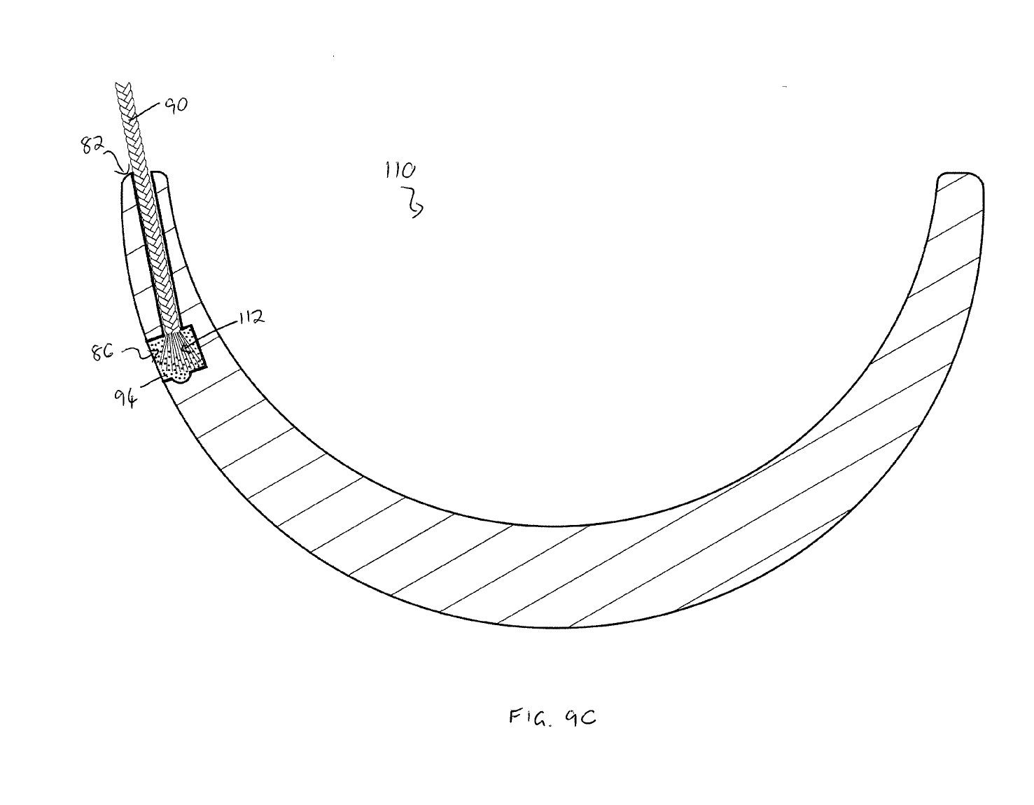

As shown in FIG. 7A a first, generally cylindrical recess 82 is formed (e.g. drilled) in an edge 84 of the cup 80 and extends deep into the cup body. In accordance with FIGS. 7B and 7C, a larger, second, generally cylindrical recess 86 is formed (e.g. drilled) from an exterior surface 88 of the cup 80 and which passes through a lower region of the first recess 82 but which does not extend into an interior of the cup 80. An end of a braided polymer cable attachment means 90 is then inserted through the first recess 82 and out of the second recess 86 as shown in FIG. 7D. A knot 92 is then tied in the end of the cable 90 and the attachment means is pulled back through the first recess 82 so that the knot 92 is located within the second recess 86 in accordance with FIGS. 7E, 7F and 7G. A polymer seal 94 is then formed around the knot 92 in the second recess 86 as shown in FIGS. 7H and 71. The exterior surface 88 of the cup 80 (including the exposed surface of the seal 94) may then be coated to promote bone ingrowth.

FIGS. 8A through 8C show a third method of manufacturing a cup 100 in accordance with an embodiment of the invention. The cup 100 is similar to the cup 80 and so like reference numerals will be employed where appropriate. In this case, the first and second recesses 82, 86 are formed as described above but the attachment means is in the form of a braided metal wire 102 and a metal ball 104 is crimped on the end of the wire 102 instead of a knot being tied. As above, a polymer seal 94 is formed around the ball 104 in the second recess 86 to fix the retaining portion of the attachment means in place.

FIGS. 9A through 9C show a fourth method of manufacturing a cup 110 in accordance with an embodiment of the invention. The cup 110 is also similar to the cup 80 and so like reference numerals will be employed where appropriate. In this case, the first and second recesses 82, 86 are formed as described above but instead of tying a knot in the end of the braided polymer cable 90 the end of the cable 90 is unravelled to form a frayed end 112. As above, a polymer seal 94 is formed around the frayed end 112 in the second recess 86 to fix the retaining portion of the attachment means in place. It is believed that this embodiment will provide a large surface area around the fibres of the frayed end 112 for the seal 94 to chemically link to, in order to securely fix the attachment means to the cup 110.

It will be appreciated by persons skilled in the art that various modifications may be made to the above-described embodiments without departing from the scope of the present invention, as defined by the claims. For example, features shown in one embodiment may be mixed and matched with features shown in other embodiments.

1. An acetabular cup prosthesis comprising:
at least one recess in an edge of the cup; and
at least one attachment means having a projecting portion for attachment of the cup to an introducer, configured for insertion of the cup into a patient, and a retaining portion for retaining the attachment means on the cup, the retaining portion being located in a respective one of said recesses and fixedly secured in said respective recess.

2. An acetabular cup prosthesis according to claim 1, wherein the retaining portion comprises an enlarged portion relative to an opening of the recess in the edge of the cup such that the enlarged portion constitutes mechanical means for fixing the attachment means to the cup.

3. An acetabular cup prosthesis according to claim 2, wherein the enlarged portion is formed by affixing a mass to an end of the retaining portion.

4. An acetabular cup prosthesis according to claim 2, wherein the enlarged portion is formed by tying a knot in an end of the retaining portion.

5. An acetabular cup prosthesis according to claim 3, wherein the attachment means comprises a metal cable and the enlarged portion is constituted by a metal ball which is crimped or welded to the metal cable.

6. An acetabular cup prosthesis according to claim 3, wherein the attachment means comprises a polymer cable and the enlarged portion is constituted by a polymer mass which is moulded to an end of the polymer cable.

7. An acetabular cup prosthesis according to claim 6, wherein the polymer cable is braided and the end of the polymer cable is unravelled prior to moulding the polymer mass to the end of the polymer cable.

8. An acetabular cup prosthesis according to claim 2, wherein an undercut is provided to accommodate the enlarged portion and retain the attachment means on the cup.

9. An acetabular cup prosthesis according to claim 8, wherein the undercut is located a distance away from the edge of the cup so as to transfer stresses transmitted through the attachment means to the cup, from a thin and more fragile edge of the cup to a thicker, more robust region of the cup.

10. An acetabular cup prosthesis according to claim 1, wherein a locking element is provided in the recess for the attachment means to lock onto.

11. An acetabular cup prosthesis according to claim 10, wherein the locking element comprises a post which the retaining portion is fixed to.

12. An acetabular cup prosthesis according to claim 8, wherein the undercut is formed by providing a second recess which extends from an exterior surface of the cup into the recess in the edge of the cup.

13. An acetabular cup prosthesis according to claim 12, wherein the second recess is larger in diameter than the recess in the edge of the cup.

14. An acetabular cup prosthesis according to claim 12, wherein a seal is provided in the second recess after the retaining portion has been located and fixed therein.

15. An acetabular cup prosthesis according to claim 14, wherein the second recess comprises a second undercut to retain the seal therein.

16. An acetabular cup prosthesis according to claim 14, wherein the seal—along with the rest of an external surface of the cup—is coated with a porous particle surface for bone ingrowth.

17. An acetabular cup prosthesis according to claim 12, wherein the edge recess and the second recess intersect to form a hole with a cross-shaped cross-section such that the second recess constitutes an undercut in relation to the edge recess and the edge recess constitutes a second undercut in relation to the second recess such that no further undercuts are required.

18. A method of manufacture of an acetabular cup prosthesis, the method comprising:
forming an acetabular cup prosthesis with at least one recess in an edge of the cup;
providing at least one attachment means having a projecting portion for attachment of the cup to an introducer, configured for insertion of the cup into a patient, and a retaining portion for retaining the attachment means on the cup;
locating the retaining portion in a respective one of said recesses in the edge of the cup; and
fixedly securing the retaining portion in said respective recess.

19. The method of claim 18 wherein the retaining portion is fixedly secured in the recess by injection moulding, compression moulding, hot-forming and/or by mechanical means.

20. The method of claim 18 wherein an undercut is formed in the recess by machining or by using a lost-wax method or similar technique.

21. The method of claim 20 wherein a ceramic piece is employed to define the shape of the recess and/or undercut and/or a locking element in the recess during manufacture of the cup.
